(19) United States
(12) Patent Application Publication
(10) Pub. No.: US 2025/0255504 A1
(43) Pub. Date: Aug. 14, 2025

(54) FLOW SENSING VASCULAR IMPLANT

(71) Applicant: KONINKLIJKE PHILIPS N.V., EINDHOVEN (NL)

(72) Inventors: Aditee KURANE, MAPLE GROVE, MN (US); Arjen VAN DER HORST, TILBURG (NL); Cedar DECKER, ALBERTVILLE, MN (US)

(21) Appl. No.: 18/850,140

(22) PCT Filed: Mar. 20, 2023
(86) PCT No.: PCT/EP2023/056992

§ 371 Date: Sep. 24, 2024

Related U.S. Application Data
(60) Provisional application No. 63/325,645, filed on Mar. 31, 2022.

(51) Int. Cl. A61B 5/026; A61B 5/00; A61B 5/0215; A61F 2/82

(52) U.S. Cl. CPC A61B 5/026; A61B 5/02158; A61B 5/6862; A61F 2/82; A61B 5/0215; A61B 2560/066; A61B 2562/0223; A61F 2250/0098

(57) ABSTRACT

A vascular therapy device (10) includes a stent (2) comprising a tube having an inner surface (3) defining a central lumen (5), the stent configured to be placed at a treatment site in a blood vessel of an associated patient; and at least one sensor (12) attached to or embedded in the inner surface of the stent.

### FIELD

The following relates generally to the vascular stent arts, blood flow sensing arts, pressure sensing arts, temperature sensing arts, stenosis sensing arts, and related arts.

### BACKGROUND

The onset of vascular disease, both arterial and vascular, often becomes a life-long chronic condition for patients, requiring continuous monitoring. Recurrence of manifestations of the disease such as plaque buildup leading to narrowing of the blood vessel lumen (referred to as stenosis) is common, usually resulting in additional invasive treatments. Early detection of recurrence of stenosis is critical in minimizing the severity of any medical interventions, which both reduces the procedural risk to the patient, and preserves future treatment options. Current monitoring and detection techniques, including symptom observation, external ultrasound and intravascular fluoroscopy, have a variety of limitations, including lack of location specificity, anatomical access limitations, procedural risk and cost.

A common therapy for patients suffering from stenosis is to receive a stent to restore adequate blood flow though the stenotic vessel region affected by the disease. Restenosis of the stent due to build-up of plaque or other clotting material inside the stent is a common occurrence given the underlying biological and lifestyle conditions that contribute to the disease. Indeed, the nature of the stent as foreign and sometimes non-biological material in the body can increase likelihood of stenotic buildup at the stent.

Restenosis is most often detected by recurrence of symptoms (pain, swelling, discoloration) reported by the patient. This form of detection is not only untimely because it requires the significant progression of the disease to trigger the development of symptoms, but it also offers little information on the location of the disease. Symptoms may have resulted from restenosis of a previously treated vessel portion, or from onset of stenosis in a previously healthy vessel portion.

Ankle-brachial blood pressure comparison, or tissue oxygenation measurement are other non-invasive, scalable, detection techniques. However, they also lack specific information on the location of the disease. For example, ankle-brachial blood pressure comparison merely can indicate at least one stenotic region is present somewhere between the ankle and brachial blood pressure measurement locations.

Other detection modalities such as external ultrasound (US) or intravascular fluoroscopy are capable of detecting restenosis prior to the onset of symptoms and provide location specificity, but the cost, procedural risk and limited anatomical access (i.e., external US is not effective for detecting stenosis in vasculature of the pelvis, for example), make these techniques ill-suited for front line detection.

The following discloses certain improvements to overcome these problems and others.

### SUMMARY

In some embodiments disclosed herein, a vascular therapy device includes a stent comprising a tube having an inner surface defining a central lumen, the stent configured to be placed at a treatment site in a blood vessel of an associated patient; and at least one sensor attached to or embedded in the inner surface of the stent.

In some embodiments disclosed herein, a vascular therapy device includes a stent comprising a tube having an inner surface defining a central lumen, the stent configured to be placed at a treatment site in a blood vessel of an associated patient; and at least one pressure sensor attached to or embedded in the inner surface of the stent, the at least one pressure sensor configured to acquire at least one pressure measurement comprising blood flow resistance.

In some embodiments disclosed herein, a vascular therapy method includes: receiving a blood flow resistance measurement caused by a stent installed in a blood vessel based on at least one measurement with one or more sensors; repeating the blood flow resistance measurement over successive sessions to generate a blood flow resistance-versus-time curve; assessing restenosis at the stent based on the blood flow resistance-versus-time curve; and outputting, on a display device, an indication of the restenosis assessment.

One advantage resides in providing real time, non-invasive monitoring of blood flow through a previously implanted stent.

Another advantage resides in early detection of disease progression and providing a treating physician with location specific information that is key to the planning of additional treatment.

Another advantage resides in monitoring and preventing restenosis at a stent implanted in a patient.

Another advantage resides in reducing a need for imaging of a potential restenosis in a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIGS. 2 and 3 show other embodiments of the device of FIG. 1.

FIG. 4 diagrammatically illustrates a method of performing a vascular therapy method using the device of FIG. 1.

### DETAILED DESCRIPTION

A concern with stents is the potential for restenosis occurring at the stent. Typically, this is monitored by techniques such as doppler ultrasound imaging. However, the stent can interfere with ultrasound image quality, and detection of buildup of material on the stent by external imaging is challenging. Intravascular ultrasound imaging may provide higher image quality, but at the cost of performing a follow-up invasive intravascular procedure on the patient.

The following discloses an alternative approach in which a sensor is embedded with the stent and remains in the patient with the stent to provide monitoring of any restenosis.

In one contemplated embodiment, a passive pressure sensor is used. Such a sensor can be advantageously highly miniaturized, e.g. on the order of microns in size, and is passive in that it is energized and operated using an externally applied magnetic field. In one specific implementation, two such sensors are placed, at the upstream and downstream ends of the stent, respectively. The pressure difference between the two sensors provides a metric of the flow resistance presented by the stent. In one approach, sensor readings are acquired immediately after the stent is placed, and at subsequent intervals such as at follow-up doctor's office visits. Restenosis would then manifest as gradually increasing flow resistance over time. In some examples, when multiple tents are placed inside a vessel, a pressure gradient between the stents can be determined. In other examples, a pressure measurement can be measured at a site of the stent, and a pressure measurement can be made with a cuff at a different site, and a resulting pressure difference can be measured to determine stenosis. In another example, an external Doppler ultrasound blood flow (Q) can be measured In combination with pressure gradients (ΔP) measurement at the stent. The actual resistance R can be computed as R=ΔP/Q.

This type of sensor could detect restenosis in another way, namely that as biological material overgrowth builds up on the inner wall of the stent and on the pressure sensors, this overgrowth could impose force (corresponding to pressure) on the sensors. Increases in a measured “pressure” corresponds to increasing restenosis. One suitable approach is to measure the sensor readings for various clinical study subjects to determine what trendlines of pressure readings (or pressure difference readings, in the two-sensor embodiment) over time correspond to significant restenosis.

To enable measurement of the pressure of the blood in the stent lumen (or to detect restenosis by the force applied by biological material overgrowth) the pressure sensor is suitably placed inside the stent, that is, on the wall of the lumen of the stent. It should be noted that pressure or flow measurement, as used herein, does not necessarily imply measurement in standard pressure or flow units, but more generally encompasses a metric measured by the sensor(s) disposed with the stent that correlates with pressure and/or flow.

While entirely passive sensors are advantageous, employing one or more active flow sensors in the stent is also contemplated. For example, sensors including storage capacitors for inductively powering the sensors for measurements are contemplated.

In another approach, the embedded sensor could be a temperature sensor, and the patient (or other subject) may be injected with a cooled saline solution or a contrast solution and uptake of the cooled saline fluid into the vessel containing the stent is then detected by a temperature shift measured by the embedded temperature sensor. Here, a time-delayed uptake is expected to correspond with increased flow resistance of the stent and hence with restenosis. In another example, a patient can breathe (cold) air, and a phase delay between a first “warm” temperature blood measurement and a second “cold” temperature blood measurement can be analyzed.

If the sensor is radiopaque (or has a radiopaque coating) then it could also serve a marker for locating the stent in imaging during stent placement. By thus replacing a conventional fluoroscopic marker, the number of components of the stent would not be increased.

The disclosed approach is suitable for use with any type of stent, including but not limited to self-expanding nitinol stents.

Stents and other vascular implants may also incorporate tabs or rings or other geometric expansions in their structure to provide improved visibility when observed under fluoroscopy. One variant embodiment contemplates the complete or partial replacement of these features with sensing elements. Elements placed at either end, both ends or along the length of the implant can measure a physiologically meaningful parameter (e.g., pressure) at specific locations within the region of the implant. Changes in that parameter could then be used to detect the early onset of disease progression (e.g., changes in blood flow indicate restenosis). Additionally or alternatively, sensors that provide positional information could be included with the stent and used to detect compression, expansion, or fracture/failure of the implanted stent. Sensors placed on the inside of the stent, e.g., attached to or embedded in the inner surface of the stent, are suitably in direct line with the blood and thus the blood-stent interface. This facilitates measuring tissue overgrowth on the inner stent surface and predicting patency loss and even intervening earlier to improve stent patency.

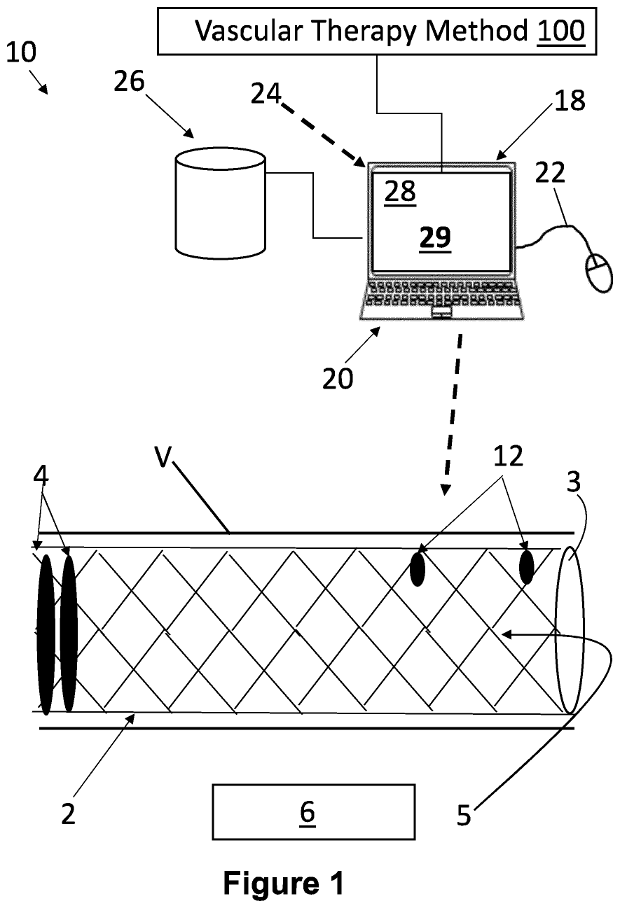

With reference to FIG. 1, an illustrative vascular therapy (i.e., thrombectomy or atherectomy) apparatus 1 is diagrammatically shown. As shown in FIG. 1, the apparatus 1 includes a therapy device 10 comprising a vascular therapy device 2 (e.g., a self-expanding stent which can be self-expanding, a self-expanding filter, and so forth) that is implanted into a blood vessel V. In some examples, the stent 2 comprises a self-expanding Nitinol stent. The stent 2 comprises a tube having an inner surface 3 that defines a central lumen 5. The stent 2 is placed at a treatment site in a blood vessel V of an associated patient.

Delivery of the stent 2 into the blood vessel V may be accomplished, for example, using a catheter or other intravascular instrument (not shown) that is inserted into a blood vessel via an incision and moved to the treatment site where the stent 2 is deployed. Various known stent delivery instrument systems can be used for this purpose. After deployment of the stent 2 into the blood vessel V, the intravascular instrument is withdrawn, leaving the deployed stent in place. In the case of a self-expanding (e.g. nitinol) stent, the stent delivery typically entails compressing the stent into a recess at or near the tip of the intravascular instrument, and releasing the stent by pushing it out of the recess using a suitable mechanism (e.g., wire- or cable-driven) of the intravascular instrument. In another approach, the stent (which in this approach may or may not be self-expanding) is deployed and an inflatable balloon at or near the tip of the interventional instrument is positioned inside the deployed stent and inflated to expand and press the stent into the wall of the blood vessel V. Typically, the stent 2 is a hollow tube which thus serves to increase the blood vessel diameter to relieve the stenosis. However, it is also contemplated that the stent 2 may optionally include other features (not shown), such as a filter disposed in the inside the central lumen 5 of the stent, or a one-way valve such as a bicuspid or tricuspid valve disposed in the inside the central lumen 5 of the stent for use in suppressing reversal of blood flow. Such a one-way valve is sometimes included in a vein of the leg, for example, to combat blood pooling in the foot or lower leg. The term stent as used herein is intended to encompass such variants.

As shown in FIG. 1, the self-expanding stent 2 can include one or more radiopaque markers 4 (two of which are shown in FIG. 1, although any suitable number of markers can be used). The radiopaque markers 4 can take any form, such as a radiopaque coating applied to some portion or all of the wires or other material making up the stent 2, and/or separate radiopaque marker elements attached to the stent by metallurgical bonds, or so forth. Such radiopaque markers 4 provide visibility of the stent 2 in a medical imaging modality (not shown) such as X-ray or computed tomography (CT) which may be used to provide visualization of the stent delivery process for placement of the stent 2 in the blood vessel V using an intravascular procedure such as one just described.

At least one sensor 12 is attached to, or embedded in, the inner surface 3 of the stent 2. In one example embodiment, the at least one sensor 12 comprises a temperature sensor configured to measure a temperature of blood flowing through the central lumen 5 of the stent 2. In another example embodiment, the at least one sensor 12 includes a fluid flow resistance sensor configured to measure blood flow resistance of the stent 2 placed at the treatment site in the blood vessel V. In some embodiments, such a fluid flow sensor may comprise two pressure sensors spaced apart along the axis of the central lumen 5 of the stent 2 to measure a metric of the fluid flow resistance as a measured pressure difference. In some examples, the at least one sensor 12 comprises an active sensor including a storage capacitor configured to inductively supply power to the at least one sensor 12. In other examples, the at least one sensor 12 comprises a passive sensor energized by an externally applied magnetic field supplied by an associated magnetic source 6. In another example, in lieu of radiopaque markers 4, the at least one sensor 12 can be coated with a radiopaque coating.

FIG. 1 further shows an electronic processing device 18, such as a workstation computer, or more generally a computer. The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The electronic processing device 18 is programmed to determine, based on measurements obtained from the at least one sensor 12, whether a blood flow resistance measured by the at least one sensor 12 underruns a predetermined fluid flow resistance threshold (i.e., stored in the non-transitory storage media 26). An indication 29 of the blood flow resistance underrunning the predetermined fluid flow resistance threshold can be output on the display device 24.

FIG. 1 shows two sensors 12, although any suitable number of sensors 12 may be implemented. In some embodiments, the sensor(s) 12 includes one or more pressure sensors 12 configured to measure the blood flow resistance caused by the stent 2 based on at least one pressure measurement acquired by the pressure sensor(s) 12. For example, the sensor(s) 12 can include a first pressure sensor 12 and a second pressure sensor 12 disposed at spaced apart locations along a central axis of the central lumen 5. Such a spaced-apart arrangement enables measurement of blood flow resistance as a pressure difference between the readings of the two pressure sensors 12. As previously noted, this blood flow resistance may not necessarily be provided in standard flow resistance units, but is a quantitative metric of the blood flow resistance.

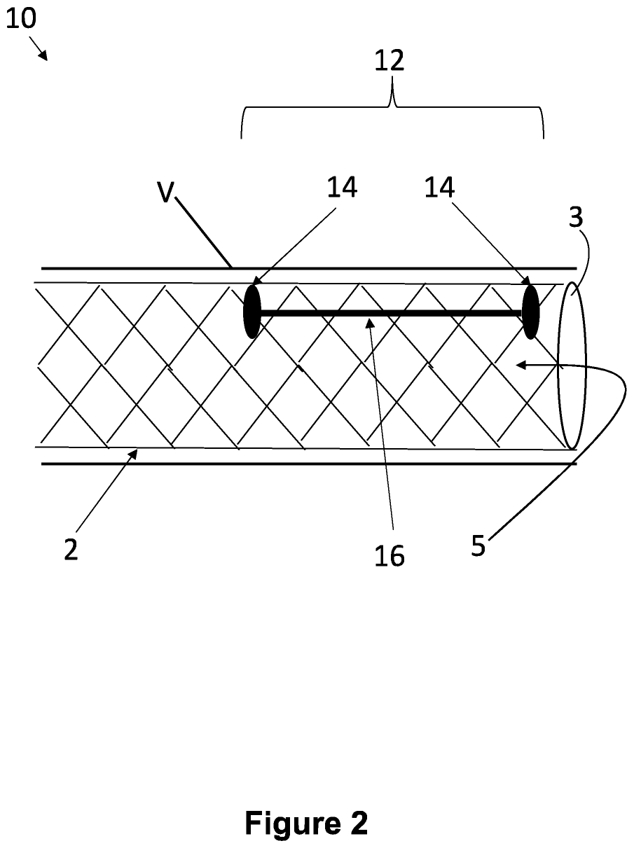

In another example, as shown in FIG. 2, each sensor 12 includes first and second permanent magnets 14 In one example, the first and second permanent magnets 14 can be connected by a coupler 16. By changing a distance between By changing the distance e.g. by a pressure sensitive membrane) the pressure and/or temperature can be measured. . . . In another example, the first and second permanent magnets 14 are positioned at opposing ends of the stent 2. One of the first and second permanent magnets 14 is fixed to the stent 2, and the other of the first and second permanent magnets 14 is configured to oscillate relative to the fixed magnet.

A measurement of the stent placed at the treatment site is obtainable from the at least one sensor element by inducing a resonant rotational oscillation of at least one of the permanent magnets using an externally applied magnetic field. Some suitable sensors of this passive dual-magnet type are described in Gleich et al., U.S. Pub. No. 2021/0244305 A1 which is incorporated herein by reference in its entirety. In one design of this type, each sensor 12 includes the two permanent magnets 14 connected by the elastic coupler 16 located inside a container (not shown) that is secured to (e.g. embedded in) the inner surface 3 of the stent 2. One magnet is held in fixed position and the other is free to oscillate with a rotational movement. (In another embodiment, both magnets may be free to oscillate). The oscillation takes place at a resonance frequency, which is a function of a distance between the magnets 14, which can be changed as a function of a measured pressure or temperature by the sensor 12. The oscillation resonance frequency can be sensed remotely by measuring a magnetic field produced by the magnetic source 6. The magnetic field is altered by the oscillation of the free magnet 14. As the magnetic source 6 may be located outside of the body of the patient (typically placed near or on the patient's skin in close proximity to the internal location of the stent 2), this measurement is entirely noninvasive.

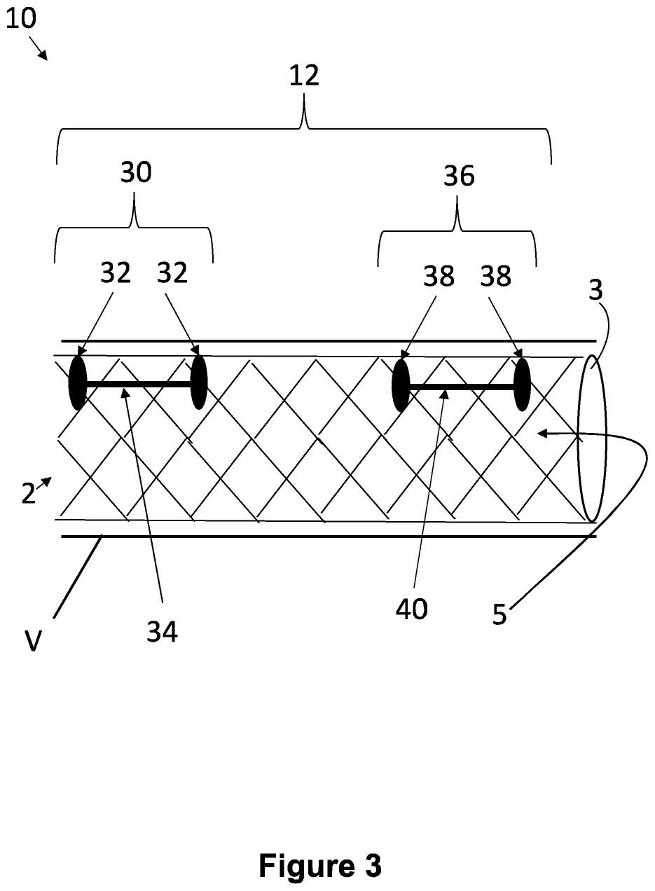

In another example, as shown in FIG. 3, the sensor(s) 12 include a first sensor element 30 of the dual-magnet type comprising first and second magnets 32 connected by an elastic coupler 34 that is attached to or embedded in a first location of the inner surface 3 of the stent 2. A second sensor element 36 of the dual-magnet type includes first and second permanent magnets 38 connected by an elastic coupler 40 that is attached to or embedded in a second location of the inner surface 3 of the stent 2. The second location is spaced apart from the first location along a central axis of the central lumen 5 of the stent 2. A pressure difference between the readings of the two sensors 30 and 36 serves as a metric of the fluid flow resistance as follows: Restenosis in the stent 2 is expected to lead to increased flow resistance-hence, an increase in the measured pressure difference over time correlates with increasing restenosis at the stent 2. In this approach, to enable the two pressure sensors 30 and 36 to be read independently, they can optionally be designed to oscillate at different resonant frequencies. This can be done, for example, by making the elastic coupler 16 and or the masses and/or magnetizations of the two permanent magnets 14 different from the two pressure sensors 30 and 36.

At each office visit, a frequency sweep of the frequency of the magnetic field produced by the magnetic source 6 is applied to identify the resonant frequency of each of the two dual-magnet pressure sensors 12. For assessing restenosis over time, in one suitable workflow such a measurement is performed at successive time intervals, for example at successive physician office visits. The pressure difference (which here is the fluid flow metric) is measured at each such visit, thereby generating a fluid flow resistance-versus-time curve over several days, week, months, or years. Based on this curve, the physician can assess the extent of restenosis. This assessment may be threshold-based, or may be based on a rate-of-increase of the flow resistance over time (i.e., a slope of the fluid flow resistance-versus-time curve). Optionally, clinical trials could be used to calibrate a relationship between the extent of restenosis and the change over time of the fluid flow resistance (or resistance slope) measured using the sensors 30, 36. In another approach, ex vivo bench trials could be conducted to calibrate the relationship, for example, using a porcine blood vessel with an instance of the stent 2 implanted therein and a mechanical pump providing flow of saline solution simulating blood flow.

These are merely examples, and should not be construed as limiting. These examples can also use temperature readings measured by the sensors 12 in lieu of pressure readings.

The at least one electronic processor 20 is configured as described above to perform a vascular therapy method or process 100. The non-transitory storage medium 26 stores instructions which are readable and executable by the at least one electronic processor 20 to perform disclosed operations including performing the vascular therapy method or process 100. In some examples, the method 100 may be performed at least in part by cloud processing.

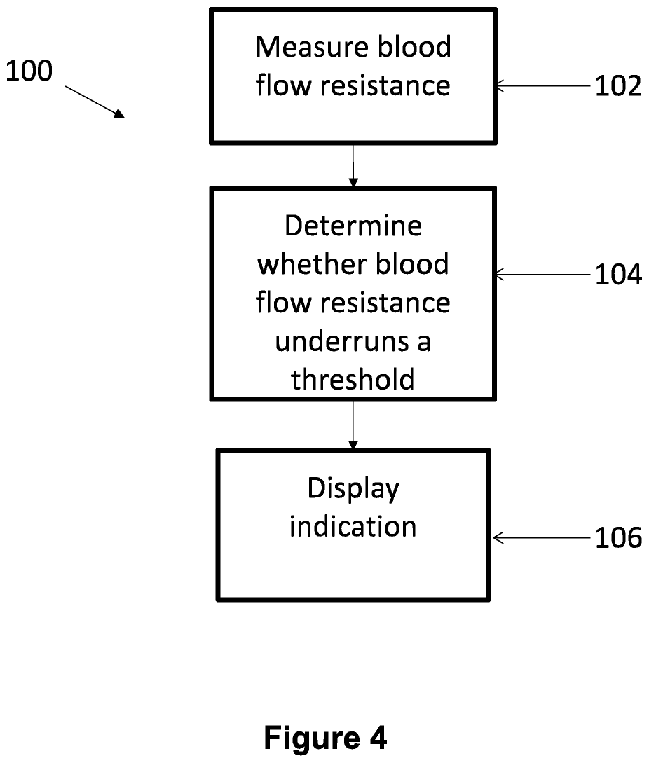

Referring to FIG. 4, and with continuing reference to FIGS. 1-3, an illustrative embodiment of the vascular therapy method 100 is diagrammatically shown as a flowchart. To begin the method 100, the stent 2 is deployed into the blood vessel V via the therapy device 10 at a treatment location in a blood vessel V of a patient.

At an operation 102, a blood flow resistance measurement caused by a stent (2) installed in a blood vessel V is measured by the sensor(s) 12 and received by the electronic processing device 18. At an operation 104, the electronic processing device 18 determined whether the blood flow resistance underruns a predetermined fluid flow resistance threshold. At an operation 106, an indication 29 of the blood flow resistance underrunning the predetermined fluid flow resistance threshold is output on the display device 24. As previously noted, other metrics could be used, such as the slope or other characteristic of a blood flow resistance-over-time curve measured over successive physician's office visits.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular therapy device, comprising:
a stent comprising a tube having an inner surface defining a central lumen, the stent configured to be placed at a treatment site in a blood vessel of an associated patient; and
at least one sensor attached to or embedded in the inner surface of the stent.

2. The vascular therapy device of claim 1, wherein the at least one sensor includes a fluid flow resistance sensor configured to measure blood flow resistance at the treatment site in the blood vessel.

3. The vascular therapy device of claim 2, further comprising an electronic processing device configured to:
determine whether the blood flow resistance underruns a predetermined fluid flow resistance threshold; and
output, on a display device, an indication of the blood flow resistance underrunning the predetermined fluid flow resistance threshold.

4. The vascular therapy device of claim 1, wherein the at least one sensor comprises:
one or more pressure sensors configured to measure the blood flow resistance caused by the stent based on at least one pressure measurement acquired by the one or more pressure sensors.

5. The vascular therapy device of claim 1, wherein the at least one sensor comprises:
a first pressure sensor and a second pressure sensor disposed at spaced apart locations along a central axis of the central lumen.

6. The vascular therapy device of claim 1, wherein:
the at least one sensor includes at least one sensor element comprising first and second permanent magnets connected by an elastic coupler;
wherein a measurement of the stent placed at the treatment site is obtainable from the at least one sensor element by inducing a resonant rotational oscillation of at least one of the permanent magnets using an externally applied magnetic field.

7. The vascular therapy device of claim 6, wherein the at least one sensor element comprising first and second permanent magnets connected by an elastic coupler includes:
a first sensor element comprising first and second permanent magnets connected by an elastic coupler that is attached to or embedded in a first location of the inner surface of the stent; and
a second sensor element comprising first and second permanent magnets connected by an elastic coupler that is attached to or embedded in a second location of the inner surface of the stent;
wherein the second location is spaced apart from the first location along a central axis of the central lumen of the stent.

8. The vascular therapy device of claim 1, wherein the at least one sensor comprises a passive sensor energized by an externally applied magnetic field supplied by an associated magnetic source.

9. The vascular therapy device of claim 1, wherein the at least one sensor comprises an active sensor including a storage capacitor configured to inductively supply power to the at least one sensor.

10. The vascular therapy device of claim 1, wherein the at least one sensor comprises a temperature sensor configured to measure a temperature of blood flowing through the central lumen of the stent.

11. The vascular therapy device of claim 10, wherein the at least one sensor comprises:
a first temperature sensor and a second temperature sensor disposed at spaced apart locations along a central axis of the central lumen.

12. The vascular therapy device of claim 1, wherein the stent comprises a self-expanding Nitinol stent.

13. A vascular therapy device, comprising:
a stent comprising a tube having an inner surface defining a central lumen, the stent configured to be placed at a treatment site in a blood vessel of an associated patient; and
at least one pressure sensor attached to or embedded in the inner surface of the stent, the at least one pressure sensor configured to acquire at least one pressure measurement comprising blood flow resistance.

14. The vascular therapy device of claim 13, further comprising an electronic processing device configured to:
determine whether the blood flow resistance underruns a predetermined fluid flow resistance threshold; and
output, on a display device, an indication of the blood flow resistance underrunning the predetermined fluid flow resistance threshold.

15. The vascular therapy device of claim 13, wherein the at least one pressure sensor comprises:
a first pressure sensor and a second pressure sensor disposed at spaced apart locations along a central axis of the central lumen.

16. The vascular therapy device of claim 13, wherein:
the at least one pressure sensor includes at least one pressure sensor element comprising first and second permanent magnets connected by an elastic coupler;
wherein a measurement of the stent placed at the treatment site is obtainable from the at least one sensor pressure element by inducing a resonant rotational oscillation of at least one of the permanent magnets using an externally applied magnetic field.

17. The vascular therapy device of claim 16, wherein the at least one pressure sensor element comprising first and second permanent magnets connected by an elastic coupler includes:
a first pressure sensor element comprising first and second permanent magnets connected by an elastic coupler that is attached to or embedded in a first location of the inner surface of the stent; and
a second pressure sensor element comprising first and second permanent magnets connected by an elastic coupler that is attached to or embedded in a second location of the inner surface of the stent;
wherein the second location is spaced apart from the first location along a central axis of the central lumen of the stent.

18. The vascular therapy device of claim 6, wherein the at least one pressure sensor comprises one of:
a passive sensor energized by an externally applied magnetic field supplied by an associated magnetic source.
an active sensor including a storage capacitor configured to inductively supply power to the at least one sensor.

19. The vascular therapy device of claim 13, wherein the stent includes one or more radiopaque markers;
wherein the electronic processing device is programmed to perform an image analysis based on a change in configuration of the one or more radiopaque markers in successive images of the time sequence of images of the stent acquired during a placement procedure of the stent within the blood vessel.

20. A vascular therapy method, comprising:
receiving a blood flow resistance measurement caused by a stent installed in a blood vessel based on at least one measurement with one or more sensors;
repeating the blood flow resistance measurement over successive sessions to generate a blood flow resistance-versus-time curve;
assessing restenosis at the stent based on the blood flow resistance-versus-time curve; and
outputting, on a display device, an indication of the restenosis assessment.
